(19) United States
(12) Patent Application Publication
(10) Pub. No.: US 2011/0281369 A1
(43) Pub. Date: Nov. 17, 2011

(54) ANALYSIS OF MYCOPHENOLIC ACID IN SALIVA USING LIQUID CHROMATOGRAPHY TANDEM MASS SPECTROMETRY

(75) Inventors: Fatemeh Akhlaghi, Wakefield, RI (US); Anisha E. Mendonza, Plainsboro, NJ (US)

(73) Assignee: Board of Governors for Higher Education, State of Rhode Island and Providence Plantations, Providence, RI (US)

(21) Appl. No.: 13/189,803

(22) Filed: Jul. 25, 2011

Related U.S. Application Data
(63) Continuation of application No. 12/164,511, filed on Jun. 30, 2008, now abandoned.
Application No. 12/164,511 is a continuation of application No. PCT/US2007/061214, filed on Jan. 29, 2007.
(60) Provisional application No. 60/762,929, filed on Jan. 27, 2006.

(51) Int. Cl. G01N 33/50

(52) U.S. Cl. 436/94; 436/93

(57) ABSTRACT

A method for mass spectrometric analysis of a saliva sample possibly containing mycophenolic acid or its metabolites mycophenolic acid phenyl glucuronide (MPAG) or mycophenolic acid acyl-glucuronide (Acyl-MPAG), including the steps: (a) providing a saliva sample containing one or more drug or metabolites; (b) deproteinating the sample; (c) separating the one or more drug or metabolites from the saliva sample; and (d) analyzing the one or more drug or metabolites using a mass spectrometer. The sample containing one or more MPA or metabolites is obtained from in an oral fluid based biological samples i.e. whole saliva or saliva obtained by chemical or mechanical stimulation or from specific salivary glands. The size of the sample contains one or more MPA or metabolites is at least about 100 microL. A kit for use in mass spectrometric analysis of a sample may contain one or more MPA or metabolites from saliva samples, comprising: (a) reagents for deproteinating of the saliva sample, including internal standards; (b) reagents for separating the one or more MPA or metabolites from the saliva sample; (c) reagents for analyzing the one or MPA or metabolites using a mass spectrometer; (d) a solution of one or more MPA or metabolites in saliva samples; and (e) instructions for analyzing the one or more MPA or saliva using a mass spectrometer. The kit includes (a) mobile phase solutions; (b) a chromatography column; and (c) a quality control specimen.

### PRIORITY INFORMATION

This application is a continuation application of U.S. patent application Ser. No. 12/164,511 filed on Jun. 30, 2008, which is a continuation application of International Patent Application No. PCT/US2007/061214, filed on Jan. 29, 2007, which claims priority to U.S. Provisional Patent Application 60/762,929 filed on Jan. 27, 2006, each of which is incorporated herein by reference in its entirety.

### BACKGROUND OF THE INVENTION

Mycophenolic acid (MPA), is an immunosuppressive agent commonly used for the prevention of organ rejection after transplantation and for the treatment of autoimmune disease including psoriasis, rheumatoid arthritis etc. It has been suggested that monitoring total or unbound concentration of MPA and adjusting the dose accordingly may improve its side effects profile including gastrointestinal side effects and leucopenia.

Saliva is an oral fluid that has been described as an “ultra-filtrate of plasma”. Saliva has recently been well established as a diagnostic tool in detecting many of the molecules that are found in plasma and at levels equivalent to those found in blood. For certain applications therefore, by testing saliva, one can obtain similar information on the status of a person as one can obtain from blood, without the need to collect a specimen invasively. Many commercial methods are now available for the salivary measurement of ethanol, drugs of abuse, cortisol, steroid hormones etc. To the inventors' knowledge however, there has not been any commercialization of the use of assay methods for the measurement of pharmacological agents in saliva.

All available technologies and assay methods to measure the concentration of MPA are using blood samples. Saliva offers a convenient procedure for sample collection. No venipuncture is required as is the case with blood collection and saliva sampling can be performed, with minimal training, by the patient or caregiver. Saliva monitoring requires small amount of sample (0.1 mL) and is ideal for drug monitoring in children and patients with difficult venous access. Drugs enter saliva predominately via passive diffusion, a process that is also limited to the unbound fraction of the drug since the “protein-bound drug complex” is unable to pass through small channels in the capillaries of salivary glands. It is therefore conceivable that the salivary concentration will reflect the unbound and pharmacologically active species of a drug.

Currently around 25,000 organ transplantations are carried out every year in the United States and approximately 70-80% of all patients remain on immunosuppressive therapy with MPA. Considering that the drug has been used since 1995, an anticipated 192,000 to 220,000 transplant recipients are receiving Cellcept®, the commercial form of MPA. A larger number of patients also receive transplantation in Europe and the rest of the world that are treated with MPA. According to Roche Laboratories 5000 prescriptions of Cellcept® is filled every week in the United States alone for transplantation and autoimmune related diseases. Each transplant recipient is likely required to be monitored for the MPA concentration once weekly for the first three months, then every month giving a conservative estimate of 20 concentrations monitored per year. Assuming that 200,000 patients may use a saliva based method for MPA measurement 20 times per year and the conservative cost of each test is $50, the estimated yearly sale of this method would be a total of $200 million per year in the US alone.

Every time a person is required to have their MPA levels tested, blood must be drawn. Also because MPA undergoes enterohepatic recirculation resulting in high concentrations approximately around 6 to 12 hours post dose, a single blood concentration obtained before the next dose is usually not enough to assess the extent of drug exposure. For this reason, usually 3-4 blood samples must be obtained in one day. If the patients were able to be tested by a saliva sample obtained via swab or passive drool, the test would be a lot less invasive and painful. The test would also be less expensive and thus save the medical community a considerable amount of money.

### SUMMARY OF THE INVENTION

An analytical method was developed and validated for quantification of salivary MPA using liquid chromatography tandem mass spectrometry (LC-MS/MS). The sample preparation included the addition of 50 μL internal standard solution (500 μg/L indomethacin (INDO) in methanol), to 100 μL saliva sample followed by the precipitation of salivary proteins using 200 μL acetonitrile. Supernatants were dried and reconstituted in 100 μL of 85:15% v/v mixture of methanol and water containing 0.05% formic acid and 20 μL was injected onto the analytical column. The mobile phase comprised of a gradient mixture of methanol and 0.05% formic acid, and the total run time was 7.5 min.

A calibration curve was prepared and found to be linear over a concentration range of 2.5-800 μg/L (r=0.9999) and the recovery was greater than 90%. The accuracy was within the ±15% limit and intra- and interday CV % ranged from 2.8-5.2% Mean±SD of saliva concentration in saliva samples from kidney transplant recipients was 31.4±32.3 μg/L 2.6-2204 μg/L; n=100) and correlated well with total or unbound concentrations of MPA.

A robust, sensitive and specific method for quantification of MPA in saliva was developed using LC-MS/MS and validated according to FDA guidelines. A simple method was devised for extraction of MPA from saliva matrix that only consists of a protein precipitation step followed by centrifugation. The method requires only 100 μL of saliva that is easily obtained by passive drool. The saliva concentration represents a free concentration of the drug.

The concentration of MPA was measured in paired saliva and plasma samples from 29 kidney transplant recipients during 12-hour dosing interval after MIPA dose. At the completion of the study, 244 saliva samples were analyzed. Overall, MPA concentrations in saliva were in good agreement with the unbound plasma concentrations. The average deviation between saliva and unbound plasma concentrations was 0.49 ng/mL however it transpires that the deviation is greater at morning trough (possibly because of the presence of blood in saliva) and during the absorption phase (possibly because of delay in distribution between plasma and saliva). Based on this preliminary clinical information, we believe saliva is a feasible specimen that allows simple and non invasive monitoring of the pharmacologically active unbound MPA. More rigorous clinical studies are required to refine the sample collection strategies i.e. to investigate the effect of food, saliva stimulation, mouth rinsing and so forth on the MPA concentration in saliva.

The long term objective was to improve immunosuppressive therapy of mycophenolic acid (MPA) by means of developing a convenient and more specific monitoring strategy for this agent. Specifically, to develop and validate a sensitive and specific analytical method for measuring MPA concentrations in saliva; to explore the association between total saliva concentration of MPA with its total and unbound plasma concentrations in renal transplant recipients who are taking MIPA as part of their maintenance immunosuppressive therapy; and to explore the factors that influence saliva to plasma ratio of MPA including serum albumin, creatinine, BUN, pH of saliva and plasma and total concentration of MPA and MPAG.

One goal was to develop a sensitive, specific, reliable and reproducible assay for quantification of MPA in saliva using liquid chromatography and tandem mass spectrometry (LC-MS/MS) and to fully validate it according to the rigorous guidelines set by the Food and Drug Administration of the United States. Previously, no other assays have been reported for either extraction of MPA from saliva matrix or its quantification in salivary extracts.

Initially two different solid phase extraction methods were tried. One of these methods utilizes C-18 extraction cartridges and has been used previously for extraction of MPA from plasma ultrafiltrate and the other was a solid phase extraction method using C-8 cartridges similar to a method previously developed for extraction of MPA and metabolites from plasma. These methods originally provided clean extracts and reasonable extraction recovery from saliva but both have failed the validation process because of unacceptable intra- and inter-day imprecision and accuracy resulted from non-reproducible recovery from saliva based quality control standards.

Finally the use of solid phase extraction cartridges was eliminated altogether and many different combinations of solvents were tried. Such methods are commonly referred to as liquid-liquid extraction. One combination has yielded the most reproducible and highest recovery so it was further pursued as the extraction method of choice. The extraction consists of precipitation of salivary proteins from 100 μL of saliva using 504 methanol and 200 μL acetonitrile followed by centrifugation and drying the supernatant. The concentration of MPA in the extract was then quantified using LC-MS/MS. In the next stage, the assay was validated according to the FDA guidelines. The Lower Limit of Quantification was 2.5 ng/mL and Limit of Detection was 1 ng/mL. The assay was linear over a working range of 2.5-800 ng/mL for MPA. The accuracy was within the ±15% limit and intra- and inter-day CV % ranged from 2.8-5.2%.

A simple, sensitive, and reproducible method for determination of MPA in saliva was developed. The assay method is now published in Therapeutic Drug Monitoring (Mendonza A E, Gohh R Y, Akhlaghi F. Analysis of mycophenolic acid in saliva using liquid chromatography tandem mass spectrometry; Ther Drug Monit 28: 402-406 (2006). This assay was then used to explore the association between the concentrations of MPA in saliva.

A kit is therefore provided for use in mass spectrometric analysis of a sample which may contain one or more MPA or metabolites from saliva samples. The kit includes (a) reagents for deproteinating of the saliva sample, including internal standards; (b) reagents for separating the one or more MPA or metabolites from the saliva sample; (c) reagents for analyzing the one or MPA or metabolites using a mass spectrometer; (d) a solution of one or more MPA or metabolites in saliva samples; and (e) instructions for analyzing the one or more MPA or saliva using a mass spectrometer. The kit also includes (a) mobile phase solutions; (b) a chromatography column; and (c) a quality control specimen.

### DESCRIPTION OF THE FIGURES

These and other features and objectives of the present invention will now be described in greater detail with reference to the accompanying drawings, wherein:

FIG. 1A is a chromatogram of MIPA, metabolites MPA-glucuronide (MAPG) and Acyl-MPAG (AcMPAG) extracted from saliva sample of a representative kidney transplant recipient;

FIG. 1B is a saliva based calibration curve for MPA;

FIG. 1C is a suppression-time profile illustrating the effect of saliva extract on the suppression of ionization of MPA and indomethacin, which indicates that a matrix dip occurs at time different from retention times of MPA or indomethacin;

FIG. 1D is an average concentration-time profile for MPA concentrations in saliva as compared with plasma and plasma ultrafiltrate from eleven stable kidney transplant recipients;

FIG. 2 is a chart of the total, unbound and saliva concentration of MPA at the morning before the dose of Cellcept®;

FIG. 3 is a chart indicating the concentration of transferrin in saliva at morning trough as compared to other times post Cellcept® dose;

FIG. 4 is a graph of the correlation between total and saliva concentration of MPA in 11 patients studied excluding morning trough levels (data are average concentrations at each sampling time point);

FIG. 5 is a graph illustrating the correlation between total and saliva concentration of MPA in 11 patients studied excluding morning trough levels (data are average concentrations at each sampling time point);

FIG. 6 is a steam and leaf plot showing deviation between saliva and unbound MPA concentrations in ng/mL; and

FIGS. 7A-7C show mean and standard error of the mean for (A) total concentration of MPA in 29 kidney transplant recipients over 12-hour dosing interval (B) concentration of transferrin and (C) deviation between saliva and unbound concentration of MPA respectively.

### DESCRIPTION OF THE INVENTION

Saliva offers a non-invasive specimen for drug analysis and may prove useful for routine therapeutic monitoring of drugs including immunosuppressive agents. Mycophenolic acid (MPA) is used as an immunosuppressant in combination with a calcineurin inhibitor and a corticosteroid for the prevention and treatment of allograft rejection. In vivo it reduces guanine nucleotide biosynthesis by inhibiting inosine 5′-monophosphate dehydrogenase (IMPDH). Mycophenolic acid exhibits variable pharmacokinetic characteristics. Monitoring MPA concentrations therefore, may serve as a guide to dose individualization, which may improve post transplant outcomes.

In plasma, MPA is highly bound to serum albumin with an average free fraction of approximately 2 to 3%. Since an unbound or free concentration of a drug represents the pharmacologically active form of the drug, monitoring unbound MPA may prove beneficial in the clinical practice. Several methods have been used to quantify unbound MPA in plasma including ultrafiltration followed by chromatographic analysis of MPA and equilibrium dialysis using radiolabelled MPA. These methods however, are laborious and require approximately 1 mL of plasma. Saliva represents a natural ultrafiltrate of plasma and salivary concentrations of drugs, in theory therefore, should represent the unbound concentration. An unstressful sampling versus venipuncture is another advantage of saliva monitoring hence allowing repeated sampling in a non medical environment. The saliva concentration represents the free concentration of the drug.

Indomethacin (INDO, Alfa Aesar) was used as the internal standard. All reagents and solvents were high performance liquid chromatography (HPLC) grade. Sub-stocks of MPA in methanol (1, 5 and 50 mg/L) were prepared and used to spike saliva. Calibrators and Quality Control standards (QCs) were prepared using pooled unstimulated whole saliva collected from at least six healthy volunteers (IRB Approval#HU0203-120). For each batch analyzed, a 7-point calibration curve (2.5, 25, 50, 100, 300, 500, 800 μg/L) of MPA in saliva was constructed using 1/x² linear regression, and in-house QCs at three concentrations (10, 200 and 600 μg/L) corresponding to low, medium and high levels. All calibrators and QCs were aliquoted into 2 mL cryovials and maintained at −20° C. until use.

Extraction of MPA from saliva was carried out by protein precipitation. Calibrators, QC's or patient samples were thawed in a shaking water bath at 37° C. for 5 min. The samples were then sonicated for 10 seconds and 100 μL was pipetted into a microcentrifuge tube, followed by the subsequent additions 50 μL methanol containing INDO (500 μg/L) and 200 μg/L acetonitrile. The tubes were vortex mixed for 90 seconds and centrifuged at 16,000 g for 5 min. The supernatants were carefully aspirated into glass culture tubes and dried at 50° C. in a centrifugal evaporator (Thermosavant Holbrook, N.Y.) after which they were reconstituted with 100 μL of 85:15% v/v of methanol and 0.05% formic acid in de-ionized water and a 20 μL aliquot was injected onto the column.

All LC-MS/MS conditions were previously described in an earlier publication (incorporated herein by reference in its entirety: Patel C G, Mendonza A E, Akhlaghi F, Majid 0, Trull A K, Lee T, Bolt D W. Determination of total mycophenolic acid and its glucuroinde metabolite using liquid chromatography with ultraviolet detection and unbound mycophenolic acid using tandem mass spectrometry. J Chromatogr B Analyt Technol Biomed Life Sci 2004; 8 13:287-94.) and used an API 2000 Mass Spectrometer (Sciex, Toronto, Canada). Because of the potential problems with in-source fragmentation of glucuronide metabolites to MPA, it was necessary to separate traces of MPA, MPAG and AcMPAG chromatographically. Analytical column was Zorbax Rx C8 (150 mm×4.6 mm, 5 μm) from Agilent Technologies (Palo Alto, Calif.) and mobile phase was a gradient mixture of methanol and deionized water containing 0.05% formic acid. Additionally, an ion-suppression test was performed to evaluate the effect of salivary proteins on the ionization of MPA and INDO. For this, a combined mixture of the analytes (1 mg/L each) in mobile phase was infused continuously onto the mass spectrometer and the residues extracted from blank saliva were injected simultaneously via a three way T-valve. In accordance with further embodiments, the ionization technique may involve any of photoionization, electrospray ionization, atmospheric pressure chemical ionization, electron capture ionization or selective ion monitoring, and may be performed in positive mode or negative mode.

The lower limit of quantification (LLOQ) and limit of detection (LOD) were defined at a signal to noise ratio of 5:1 and 3:1, respectively. The recovery of the extraction procedure was done by comparing the peak areas obtained from an extracted saliva based standard of MIPA or INDO with the peak areas of these analytes in methanol. To evaluate intraday coefficient of variation (CV %) of the assay, QCs were analyzed six times on the same day. Interday CV % and accuracy was evaluated by measuring the QC concentrations over 10 days using a separate calibration curve for each set. Stability studies were carried out at 10 and 600 μg/L MPA in triplicate. For short term stability studies, samples were kept on the bench top for 5 hours at room temperature and for freeze-thaw stability studies, samples were subjected to three cycles of freezing at −20° C. and thawing unassisted at room temperature. To evaluate autosampler stability, dried and reconstituted extracts were kept in the autosampler for 14-hours and then analyzed. To determine stock solution stability, methanolic based stock solutions of MPA and INDO were kept at room temperature for 8 hours and the analyte loss was compared against freshly prepared samples.

Upon obtaining IRB approval and informed consent (IRB#0159-03- and 0174-04), parallel—blood and saliva samples were collected immediately before the morning MPA dose and at 1, 2, 3, 4, 5, 7, 9, 10 and 12 hours after the MPA dose from eleven kidney transplant recipients at Rhode Island Hospital (Providence, R.I.). Patients were receiving 1000-2000 mg/day mycophenolate mofetil (Cellcept® Roche Laboratories). Unstimulated saliva samples were collected by passive drool into a plastic cup within a 5-min period of blood collection and stored at −80° C. until analysis. The patients remained fasted for the first 2 hours of sampling but then were allowed standard hospital meals. Total and unbound concentrations of MPA were measured using HPLC-UV and ultrafiltration followed by LC-MS/MS, respectively.

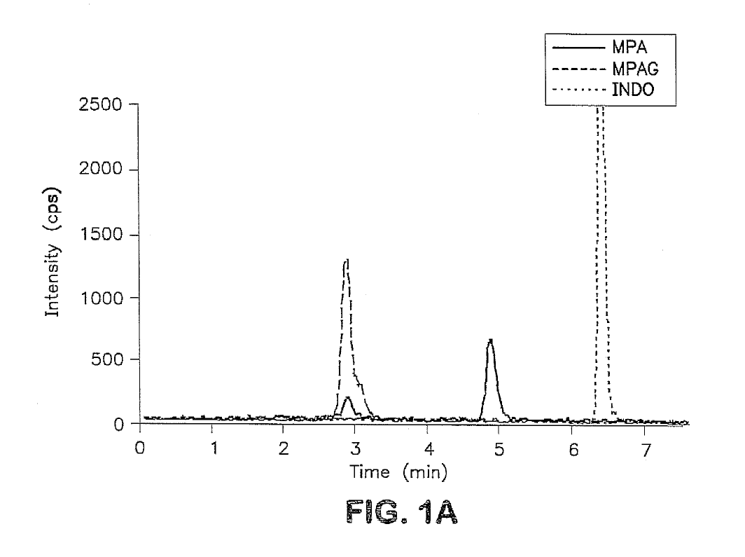

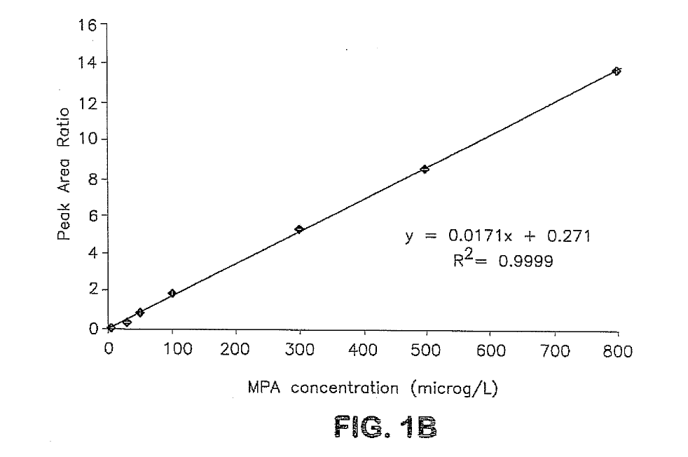

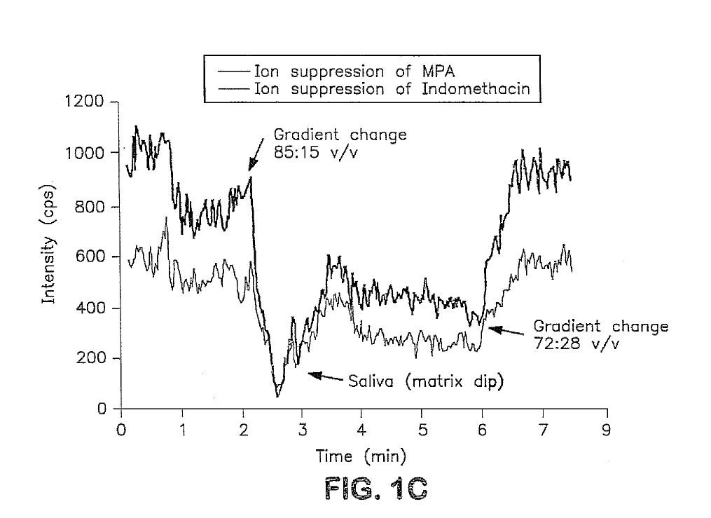

A typical chromatogram of MPA extracted from saliva obtained from a kidney transplant recipient is shown in FIG. 1A indicating a peak that was well separated from the MPAG peak. The chromatogram of MPA, metabolites MPA-glucuronide (MAPG) and Acyl-MPAG (AcMPAG) were extracted from a saliva sample from a representative kidney transplant recipient. The analytes were detected in the negative ion mode using the mass transitions of m/z 319.0→190.8 for MPA, m/z 355.9→312.2 for indomethacin and m/z 495.0 m/z 319.2 for both MPAG and AcMPAG. Some degree of in-source fragmentation of MPAG to MPA was observed hence the chromatogram shows traces of MPA at MPAG retention time however no AcMPAG peak was observed in any of the patient saliva analyzed. The LLOQ was 2.5 μg/L and LOD was 1 μg/L. The assay was linear over a working range of 2.5-800 μg/L for MPA as shown in FIG. 13 is a saliva based calibration curve for MPA.

Ion suppression studies revealed that the time of matrix or water dips did not interfere with the elution times of MPA and INDO as illustrated in FIG. 1C where an extract illustrating the effect of saliva extract on the suppression of ionization of MPA and indomethacin indicates that matrix dip occurs at times different from retention times of MPA or indomethacin.

The overall performance of the assay is shown in Table 1. The accuracy was within the ±15% limit and intra- and interday CV % ranged from 2.8-5.2%. The recovery of MPA from saliva samples were greater than 90% and for INDO was 96.0±1.5%. The results of the stability studies indicate that MPA is stable in saliva based standards under the experimental condition described above. The loss of analytes at room temperature from methanolic stock solutions of MPA and INDO was 0.6% and 10%, respectively.

TABLE 1
Results of MPA recovery, accuracy, intra- and interday precision and stability studies for MPA using saliva
based quality control standards

| Concentration | Recovery | Accuracy % | Intraday CV % | Interday CV % | Freeze-thaw | Short term | Autosampler |
|---|---|---|---|---|---|---|---|
| (μg/L) | (n = 6) | (n = 10) | (n = 6) | (n = 10) | (n = 3) | (n = 3) | (n = 3) |
| 10 | 91.3 ± 4.7 | 99.8 ± 5.2 | 2.8 | 5.2 | 103.6 ± 7.1 | 106.0 ± 3.6 | 95.0 ± 6.6 |
| 200 | 92.7 ± 3.0 | 99.8 ± 7.7 | 3.4 | 4.1 | NA | NA | NA |
| 600 | 94.8 ± 1.3 | 99.4 ± 6.9 | 3.4 | 3.6 | 98.9 ± 1.1 | 98.7 ± 2.7 | 98.2 ± 0.6 |

% CV: coefficient of variation, all plus-minus data are mean ± SD

FIG. 1D depicts the average MPA concentrations over a 12-hour dosing interval in saliva and the total and unbound concentrations in plasma from eleven kidney transplant recipients. More specifically, FIG. 1D shows an average concentration-time profile for MPA concentrations in saliva as compared with plasma (total concentration) and plasma ultrafiltrate (unbound concentration) from eleven stable kidney transplant recipients (error bars represent standard error of the mean). Mean±SD of saliva concentration was 31.4±32.3 μg/L (range: 2.6-220.4 μg/L, n=100). Salivary concentration of MPA before administration of Cellcept® morning dose was remarkably higher than saliva concentrations at other times with a considerable variability (79.8±63.7 μg/L). With the exception of morning trough, the average salivary concentrations of MPA was well correlated with its total (R²=0.826) or unbound concentration (R²=0.827) at other times.

The LC-MS/MS method described herein is a highly reliable, simple and sensitive assay requiting a small volume of saliva. Initially when a previously reported solid phase extraction procedure for MPA extraction from saliva was used, poor and non reproducible recovery was experienced. The aim was to eliminate the need for a lengthy extraction process yet provide a simple reproducible protein precipitation process rendering consistent and high recoveries for both MPA and INDO. It was also found that it is essential to break salivary protein aggregates by sonication of saliva samples before extraction. The assay was sensitive in quantifying MPA concentrations in saliva during a 12-hour dosing interval and have met FDA guidelines at all levels.

Because of its non invasive collection method, saliva monitoring of drugs and hormones have gained considerable importance. The collection method is less stressful for adults and children and can be conducted in the convenience of ones home, without the need for trained personnel. Furthermore, multiple saliva samples can be obtained at regular intervals to allow estimation of abbreviated or full area under the concentration-time curves, and the multiple samples may be analyzed simultaneously or sequentially. The distribution of drugs into saliva is dependent on factors such as degree of plasma protein binding, molecular weight, lipid solubility, ionization and salivary pH. The degree of ionization of a substance would determine if saliva to plasma ratio remains unaffected by saliva pH for instance, saliva to plasma ratio of neutral drugs or those pKa below 5.5 or above 8.5 should not be affected by salivary pH variation. The pKa value for MPA is 4.5 such that it was predicted that changes in salivary pH would not influence its saliva to plasma concentration ratio.

Disadvantages of salivary drug monitoring include possible contamination, with food particles and blood, and difficulty in pipetting due to the viscosity of saliva. The contamination problem may be alleviated by asking the donor to rinse their mouth prior to saliva collection and the viscosity problem may be resolved by using a sonifier to breakup salivary mucin. Exceptionally high morning trough concentrations in the saliva were obtained when compared with the rest of the time points. The reason could be that the patients, after overnight fasting, were experiencing dry mouth leading to more concentrated saliva. Also teeth brushing and flossing may led to some degree of bleeding and contamination of saliva with blood samples possibly resulting in high concentrations at this time point.

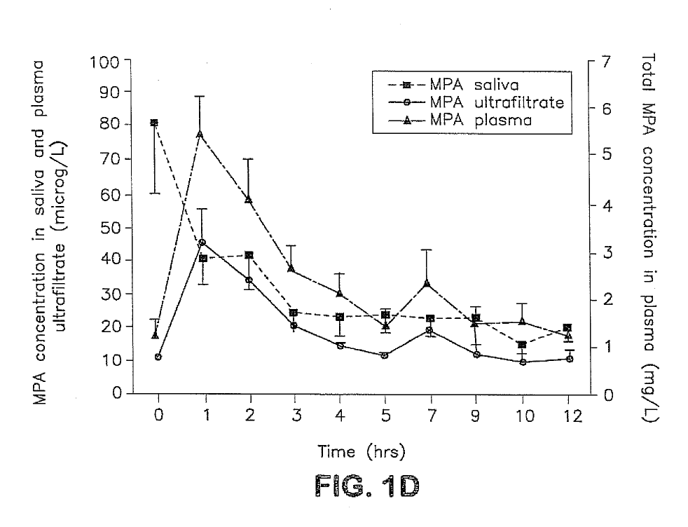

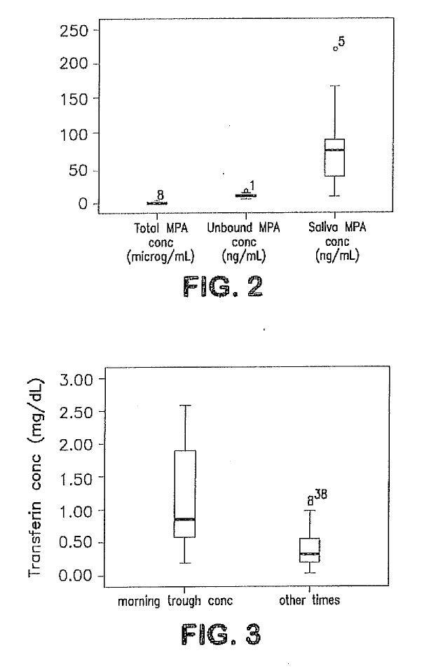

To further explore the association between the total saliva concentration of MPA with its total unbound plasma concentrations in renal transplant recipients who are taking MPA as part of their maintenance immunosuppressive therapy and to investigate the factors that influence saliva to plasma ratio of MPA, 244 paired saliva and plasma samples were collected. In the initial group of patients (11 patients, 100 samples), the Mean±SD of saliva concentrations was 31.4±32.3 μg/L (range: 2.6-220.4 μg/L). Surprisingly, salivary concentration of MPA before administration of Celcept® morning dose was remarkably higher than saliva concentrations at other times with a considerable variability (79.8±63.7 μg/L). FIG. 2 shows the concentration of MPA at trough in saliva in comparison with its total or unbound concentrations in the 11 kidney transplant recipients initially studied. The high concentration of MPA at trough could be attributed to the fact that the patients, after overnight fasting, were experiencing dry mouth leading to more concentrated saliva. Also teeth brushing and flossing may led to some degree of bleeding and contamination of saliva with blood samples possibly resulting in high concentrations at this time point.

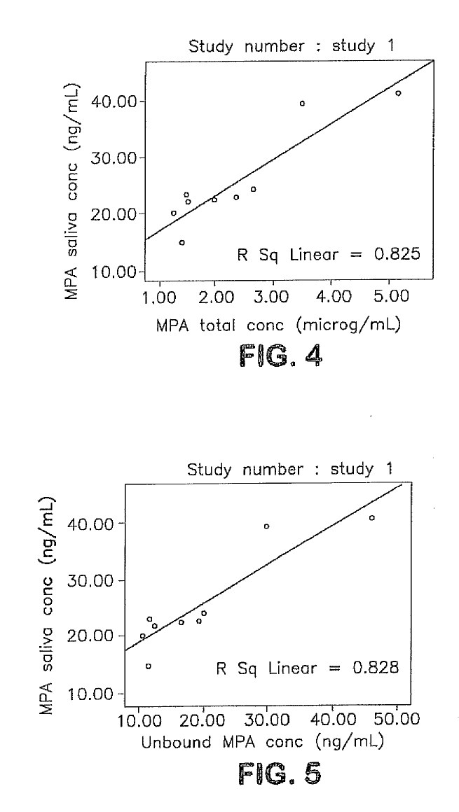

The possibility of blood leakage into saliva was measured by measuring salivary concentration of transferrin using a commercially available kit from Salimetrics LLC (State College, Pa.). This salivary blood contamination enzyme immunoassay kit measures transferrin, a large protein (mol weight 76,000) that is present in abundance in blood but normally is present in trace amounts in saliva. The manufacturer of this technique recommends that values greater than 1 mg/dL salivary transferrin should be considered as candidate for exclusion for other salivary tests. FIG. 3 depict median concentration of transferrin in saliva at morning trough as compared to other times post MPA dose indicating that high MPA concentrations observed in saliva at morning trough is most probably resulted from leakage of blood or plasma into saliva. Exclusion of the trough concentrations has resulted in a reasonably well correlation between the average total plasma (FIG. 4) or unbound (FIG. 5) concentrations with salivary concentrations of MPA.

Given the fact that the possibility of blood leakage in saliva is high at morning trough (FIG. 3), have collected 144 additional saliva samples obtained during another pharmacokinetic study. Therefore the data represented in the next section were obtained from 29 kidney transplant recipients throughout a 12-hour dosing interval. The demographic characteristics of the patient population are shown in Table 2.

TABLE 2
The demographic characteristics of the patient population

| | |
|---|---|
| Number of patients | 29 |
| Number of saliva samples per | 8-10 |
| patient | |
| Gender (M/F) | 20/0 |
| Diabetic (yes/no) | 15/14 |
| Calcineurin inhibitor- | 8/21 |
| (Cyclosporine/tacrolimus) | |

| Parameter | Mean ± SD | Range |
|---|---|---|
| Age (years) | 48.9 ± 11.9 | 18-63 |
| Weight (kg) | 86.8 ± 17.4 | 57-134 |
| Time post transplant (days) | 992.5 ± 797.0 | 132-2744 |
| Serum Creatinine (mg/dL) | 1.52 ± 0.47 | 0.80-2.70 |
| Total Protein (g/dL) | 6.8 ± 0.42 | 5.80-7.60 |
| Albumin (g/dL) | 4.26 ± 0.29 | 3.60-4.80 |
| Cholesterol (mg/dL) | 174 ± 33 | 121-253 |
| Triglyceride (mg/dL) | 178 ± 122 | 62-670 |
| Hb1Ac % | 6.87 ± 1.85 | 4.00-11.80 |

Table 3 below illustrates the saliva transferrin concentration, pH and the concentrations of total and unbound MIPA, MIPAG and Acyl-MPAG in plasma, concentration of MPA in saliva and deviation between unbound and saliva concentrations.

| | N | Mean ± SD | Median | Min-Max |
|---|---|---|---|---|
| Transferrin Concentration | 243 | 0.5 ± 0.6 | 0.3 | 0.0-6.2 |
| (mg/dL) | | | | |
| Transferrin Concentration | | | | |
| >1 mg/dL | 28 | 0.34 ± 0.22 | 0.28 | 0.02-0.99 |
| ≦1 mg/dL | 215 | 1.80 ± 1.13 | 1.44 | 1.01-6.17 |
| Saliva pH | 244 | 7.5 ± 0.7 | 7.7 | 4.5-8.6 |
| Total MPA conc (mg/L) | 244 | 3.7 ± 4.7 | 2.3 | 0.3-38.5 |
| MPAG conc (mg/L) | 244 | 51.8 ± 31.6 | 43.8 | 10.6-180.5 |
| AcMPAG conc (mg/L) | 244 | 1.8 ± 1.4 | 1.5 | 0.4-8.5 |
| Unbound MPA conc (μg/L) | 243 | 27.9 ± 36.6 | 14.5 | 1.8-267.0 |
| Saliva MPA conc (μg/L) | 246 | 28.3 ± 32.3 | 18.1 | 1.4-283.9 |
| Deviation between saliva | 243 | 0.49 ± 44.25 | 1.89 | −208.10 to 264.54 |
| and unbound conc (μg/L) | | | | |

On average the concentration of MPA measured in saliva was fairly close to the unbound concentration (Table 3). Transferrin concentration ranged from undetectable to 6.2 mg/dL but only 28 samples (11.5%) showed concentrations higher than 1 mg/dL, 15 of which occurred at morning trough. In addition saliva pH values were relatively consistent with an average of 7.5±0.7 (SD). The pKa of MPA is 4.5 which is outside the observed saliva pH values and the saliva concentrations of MPA did not show a considerable association with saliva pH (correlation coefficient=0.105; P=0.103).

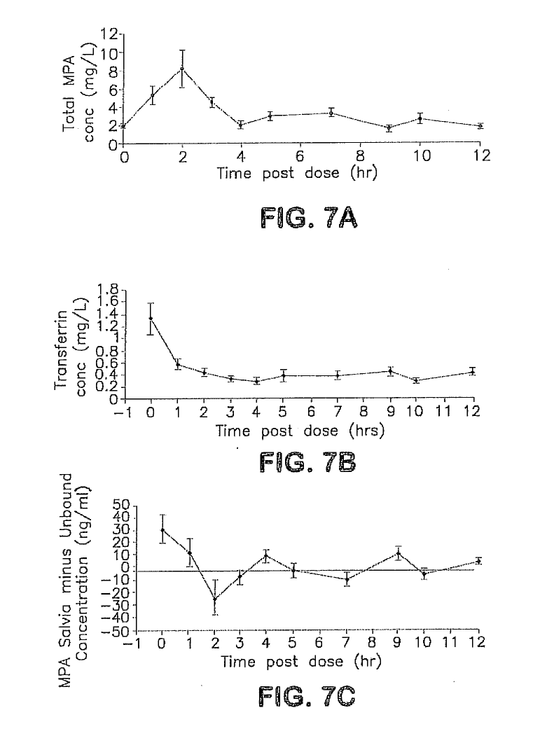

To explain factors influencing the difference in the saliva and unbound concentration of MPA, the difference between saliva and unbound concentrations of MPA was calculated and this was used to explain the sources of deviation between saliva and unbound concentrations (Table 3). The average deviation was 0.49 (μg/L) and the median was 1.89 (μg/L) but as shown in FIG. 7, there were a number of outliers both with negative and positive values.

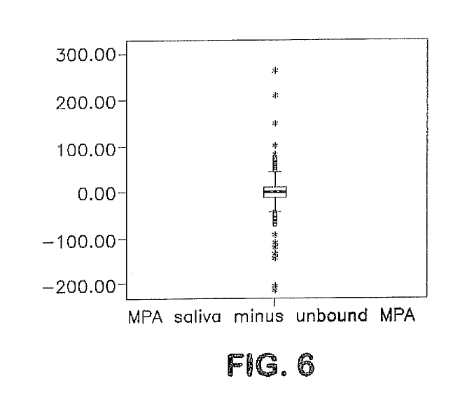

FIG. 6 shows a steam and leaf plot showing deviation between saliva and unbound MPA concentrations in (μg/L).

Table 4 shows a linear regression analysis with deviation from unbound concentration as a dependent variable and total MPA, MPAG, Acyl MPAG concentrations as well as saliva PH, transferrin concentration and patient's age as independent variables. It appears that only total MPA concentrations and transferrin levels and to a lesser extent patient age are important factors associated with the deviation between saliva and unbound concentrations.

TABLE 4
Results of linear regression analysis with deviation between
saliva and unbound concentration as dependent variable.

| | t-value | Sig. |
|---|---|---|
| (Constant) | −1.79 | 0.07 |
| Total MPA conc (mg/L) | −7.42 | 0.00 |
| MPAG conc (mg/L) | 1.49 | 0.14 |
| AcMPAG conc (mg/L) | −1.67 | 0.10 |
| Transferrin conc (mg/dL) | 6.31 | 0.00 |
| Saliva pH | 1.17 | 0.24 |
| Patient age (yr) | 1.83 | 0.07 |

Dependent Variable: Deviation between MPA concentration in saliva unbound MPA in plasma

FIGS. 7 A-C depicts the mean and standard error of total MPA concentration in plasma, saliva transferrin levels and deviation between saliva and unbound concentrations of MIPA over the 12-hour period post dose. It shows that saliva transferrin is at the highest level in morning trough samples resulting in a significantly higher MPA concentrations in saliva but it is lowered to normal levels (<0.5 mg/dL) after 2-hours post dose. Considering that all patients were reporting to the hospital in fasting state and were required to remain fasted for 2-hour, in can be speculated that the high transferrin levels in the morning are a result of tooth brushing so rinsing the mouth or eating/drinking may remedy the blood contamination problem in the majority of patients.

On the contrary, saliva MPA at two hours post close was considerably lower (FIG. 7C) than the unbound plasma concentration. MPA is rapidly absorbed in the first two hours after oral administration therefore its total or unbound concentrations in plasma rapidly change during the absorption phase. Saliva production and renewal however follows a much more static process than blood circulation hence appearance of a drug in saliva may be somewhat delayed during the absorption phase. The deviation may also relate to a patient's age (P=0.07, Table 3) because older patients have lower salivary flow and are more likely to suffer from gum disease. Stimulation of salivary flow may facilitate production and regeneration of saliva hence may reduce the time required to achieve plasma and saliva equilibrium.

A method for quantification of MPA concentrations in saliva was developed using Liquid Chromatography-Tandem Mass Spectrometry (LC-MS/MS). The method was fully validated according to the bioanalytical method development guidelines set forth by the FDA. The simple method was employed to extract MPA from saliva matrix which is an important advantage of the method. The Lower Limit of Quantification (LLOQ) of the assay is 2.5 (μg/L) with a signal to noise ratio of 10 to 1 and Limit of Detection is 1 ng/mL. With few exceptions, all observed concentrations in saliva were above the LLOQ. The assay was linear over a working concentration range of 2.5-800 ng/mL for MIPA. The accuracy was within the ±15% limit and intra- and inter-day CV % ranged from 2.8-5.2%. Initially, MIPA concentrations were measured in 11 kidney transplant recipients (100 samples). It was observed that saliva and, unbound concentrations are closely related but saliva concentrations at trough are considerably higher than unbound concentrations. 144 extra samples were collected during the 12-hour dosing interval instead of trough concentrations as was originally proposed. Overall salivary concentrations of MPA are closely related to its plasma unbound concentration with an average deviation of 0.49 (μg/L). It appears that deviation between unbound MPA and saliva concentrations is related to total MIPA concentrations, transferrin and to a lesser extent patient's age. Saliva concentration overestimates the unbound concentration at morning trough because of the presence of blood in saliva. Saliva concentration underestimates the unbound concentration during the absorption phase probably because of the fact that distribution of MPA into saliva is dynamically slower than the blood distribution.

The method may also be used in a kit for use in mass spectrometric analysis of a sample which may contain one or more MPA or metabolites from saliva samples. The kit includes (a) reagents for deproteinating of the saliva sample, including internal standards; (b) reagents for separating the one or more MPA or metabolites from the saliva sample; (c) reagents for analyzing the one or MPA or metabolites using a mass spectrometer; (d) a solution of one or more MPA or metabolites in saliva samples; and (e) instructions for analyzing the one or more MPA or saliva using a mass spectrometer. The kit also includes (a) mobile phase solutions; (b) a chromatography column; and (c) a quality control specimen.

In light of the foregoing, it will now be appreciated by those skilled in the art that numerous modifications to the disclosed embodiments are possible. It is our intention to cover these and any other changes or modifications encompassed within the scope of the appended claims.

What we claim is:

1. A method for mass spectrometric analysis of a saliva sample, containing mycophenolic acid or its metabolites mycophenolic acid phenyl glucuronide (MPAG) or mycophenolic acid acyl-glucuronide (Acyl-MPAG), comprising the steps: (a) providing a saliva sample containing one or more drugs or metabolites; (b) deproteinating the sample; (c) separating the one or more drug or metabolites from the saliva sample; and (d) analyzing the one or more drug or metabolites using a mass spectrometer.

2. The method according to claim 1 wherein the sample containing one or more MPA or metabolites is obtained from an oral fluid based biological samples, such as whole saliva or saliva obtained by chemical or mechanical stimulation or from specific salivary glands.

3. The method according to claim 1 wherein size of said sample containing one or more MPA or metabolites is at least about 100 microL.

4. The method according to claim 1 wherein said step of deproteinating the sample comprises: (a) sonicating of the samples (b) adding acetonitrile, methanol or both containing internal standards; (c) vortexing the sample; and (d) subjecting the sample to centrifugation.

5. The method according to claim 1 wherein said step of deproteinating the sample comprises subjecting the sample to precipitation with an agent containing internal standards, said agent selected from the group consisting of methanol, ethanol and salt.

6. The method according to claim 1 wherein said step of separating the one or more MPA or metabolites from the sample comprises introducing the sample to a liquid chromatography apparatus and subsequently eluting the MPA or metabolites from the column.

7. The method according to claim 6 wherein said step of separating the one or more MPA or metabolites from the sample comprises the use of a C-8 column.

8. The method according to claim 1 wherein said step of separating the one or more MPA or metabolites from the sample comprises the use of a combined liquid chromatography spectrometry apparatus.

9. The method according to claim 8 wherein the one or more MPA or metabolites are introduced into a mass spectrometer directly after being separated from the saliva sample by way of an on-line extraction and use of a built-in switch valve.

10. The method according to claim 1 wherein the mass spectrometer is a liquid chromatography-tandem-mass spectrometer.

11. The method according to claim 10 wherein the liquid chromatography-tandem mass spectrometer is equipped with an electrospray ionization source.

12. The method according to claim 1 wherein said step of analyzing the one or more MPA or metabolites using a mass spectrometer comprises an ionization technique selected from the group consisting of photoionization, electrospray ionization, atmospheric pressure chemical ionization, and electron capture ionization.

13. The method according to claim 12 wherein said ioniation technique is electro spray ionization.

14. The method according to claim 12 wherein said ionization is performed in positive mode.

15. The method according to claim 12 wherein said ionization is performed in negative mode.

16. The method according to claim 1 wherein said step of analyzing the one or more MPA or metabolites using a mass spectrometer comprises multiple reaction monitoring.

17. The method according to claim 1 wherein said step of analyzing the one or more MPA or metabolites using a mass spectrometer comprises selected ion monitoring.

18. The method according to claim 1 wherein the sample comprises a plurality of MPA or metabolites hormones and they are analyzed simultaneously.

19. The method according to claim 1 wherein the sample comprises a plurality of MPA or metabolites and they are analyzed sequentially.

20. A method of instructing an analysis of a sample that possibly contains one or more MPA or metabolites, the method comprising providing instructions to prepare the sample according to steps (b) and (c) of claim 1 and analyze the one or more MPA or metabolites from the sample according to step (d) of claim 1.

21. A kit for use in mass spectrometric analysis of a sample possibly containing one or more MPA or metabolites from saliva samples, comprising: (a) reagents for deproteinating of the saliva sample, including internal standards; (b) reagents for separating the one or more MPA or metabolites from the saliva sample; (c) reagents for analyzing the one or MPA or metabolites using a mass spectrometer; (d) a solution of one or more MPA or metabolites in saliva samples; and (e) instructions for analyzing the one or more MPA or saliva using a mass spectrometer.

22. The kit according to claim 21 further comprising: (a) mobile phase solutions; (b) a chromatography column; and (c) a quality control specimen.
